(19) United States
(12) Patent Application Publication
(10) Pub. No.: US 2022/0130550 A1
(43) Pub. Date: Apr. 28, 2022

(54) METHOD OF OBTAINING ADVICE DATA OF PHYSIOLOGICAL CHARACTERISTICS FOR A PATIENT IN ORDER TO LOWER RISK OF THE PATIENT ENTERING A MEDICAL EMERGENCY STATE

(71) Applicant: YEN-PIN CHEN, KAOHSIUNG CITY (TW)

(72) Inventors: YEN-PIN CHEN, KAOHSIUNG CITY (TW); YI-YING CHEN, TAIPEI CITY (TW)

(21) Appl. No.: 17/306,130

(22) Filed: May 3, 2021

(30) Foreign Application Priority Data
Oct. 23, 2020 (TW) 109136921

(51) Int. Cl. G16H 50/30; A61B 5/00; G16H 50/20; G16H 30/40

(52) U.S. Cl. CPC G16H 50/30; A61B 5/7267; G16H 30/40; G16H 50/20; A61B 5/7275

(57) ABSTRACT

A method of obtaining advice data of physiological characteristics for a test patient is provided to use training data pieces that include physiological data pieces of multiple reference patients to build a prediction model. The prediction model is used to calculate a probability of a test patient entering a medical emergency state based on a physiological data piece of the test patient. When the probability is greater than a threshold, a backpropagation algorithm related to the prediction model is used to acquire a target physiological data piece for the test patient to achieve, in order to lower the risk of the test patient entering the medical emergency state.

### CROSS-REFERENCE TO RELATED APPLICATION

This application claims priority of Taiwanese Invention Patent Application No. 109136921, filed on Oct. 23, 2020.

### FIELD

The disclosure relates to an assistive method for lowering the risk of a patient entering a medical emergency state.

### BACKGROUND

At present, our society has entered a mature stage of IT (information technology) development, and at the same time, IT has been integrated with various industries, such as Industry 4.0, automated driving, and various machine learning models in the medical industry. To train a machine learning model for medical use, a plurality of training data pieces that are medical data pieces respectively related to a plurality of patients are prepared and stored in a database. Each training data piece includes physiological data that is related to physiological characteristics of the corresponding patient, and that includes, for example, height, weight, age, sex, heart rate, blood pressure, etc., and a reference indication value indicating a truth (which is provided from a database) of whether the patient was transferred to an intensive care unit within a predetermined time interval counting from the time the physiological data was generated. The training data pieces are fed into a machine learning algorithm to build a prediction model that can calculate a probability of a given patient being transferred to an intensive care unit in a time interval in the future or future time interval for short. When a doctor receives physiological test data related to a test patient's physiological characteristics, the doctor can use the prediction model to generate a probability of the test patient being transferred to the intensive care unit in that future time interval based on the physiological test data.

When the test patient's probability of being transferred to an intensive care unit in the future time interval is high, the doctor may try to give the test patient appropriate treatment recommendations to reduce that probability. Conventionally, since a doctor only has their knowledge to rely on when determining what factors may affect the probability mentioned above, such as low blood pressure, low blood oxygen saturation, etc., an inexperienced doctor may not be able to make practical treatment recommendations to reduce the chances of the patient developing severe illness, and thereby miss the golden period where medical intervention could potentially prevent the test patient from being admitted to the intensive care unit.

### SUMMARY

Therefore, the object of this disclosure is to provide a method of obtaining advice data of physiological characteristics for a test patient to lower the risk of the test patient entering a medical emergency state.

According to the disclosure, the method includes steps of: A) providing a plurality of training data pieces to a computing device, wherein the training data pieces are respectively related to a plurality of reference patients, and each of the training data pieces includes: a reference physiological data piece that is related to physiological characteristics of the corresponding one of the reference patients, and a reference indication value that indicates a truth of whether the corresponding one of the reference patients entered the medical emergency state within a predetermined time interval counting from the time the reference physiological data piece of the training data piece was generated; B) by the computing device, using a machine learning algorithm that is related to a backpropagation algorithm to establish, based on the reference physiological data piece and the reference indication value of each of the training data pieces, a prediction model that uses a given physiological data piece that is related to physiological characteristics of a given patient to calculate a probability of the given patient entering the medical emergency state within the predetermined time interval counting from the time the given physiological data piece was generated; C) providing a test physiological data piece to the computing device, wherein the test physiological data piece is related to the physiological characteristics of the test patient; D) by the computing device, making the test physiological data piece serve as the given physiological data piece, and using the prediction model to calculate a test probability, which is an estimated probability of the test patient entering the medical emergency state within the predetermined time interval counting from the time the test physiological data piece was generated; E) by the computing device, determining whether the test probability is greater than a predetermined threshold; and F) by the computing device, upon determining that the test probability is greater than the predetermined threshold, using the backpropagation algorithm to acquire, based on a predetermined probability, the test physiological data piece and the prediction model, a target physiological data piece that is related to the physiological characteristics the test patient should achieve in order to lower risk of entering the medical emergency state, and generating a first suggestion message that includes the target physiological data piece and that serves as the advice data.

### BRIEF DESCRIPTION OF THE DRAWINGS

Other features and advantages of the disclosure will become apparent in the following detailed description of the embodiment(s) with reference to the accompanying drawings, of which:

FIG. 1 is a flowchart illustrating steps of a first embodiment of a method of obtaining advice data of physiological characteristics for a test patient according to this disclosure;

FIG. 2 is a block diagram illustrating a computing device to implement embodiments of this disclosure;

FIG. 3 is a flow chart illustrating step 25 of the first embodiment in detail;

FIG. 4 is a flow chart illustrating steps of a second embodiment of a method of obtaining advice data of physiological characteristics for a test patient according to this disclosure;

FIG. 5 is a flow chart illustrating step 31 of the second embodiment in detail;

FIG. 6 is a flow chart illustrating step 32 of the second embodiment in detail;

FIG. 7 is a flowchart illustrating steps of a third embodiment of a method of obtaining advice data of physiological characteristics for a test patient according to this disclosure;

FIG. 8 is a flow chart illustrating step 41 of the third embodiment in detail; and

FIG. 9 is a flow chart illustrating step 42 of the third embodiment in detail.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

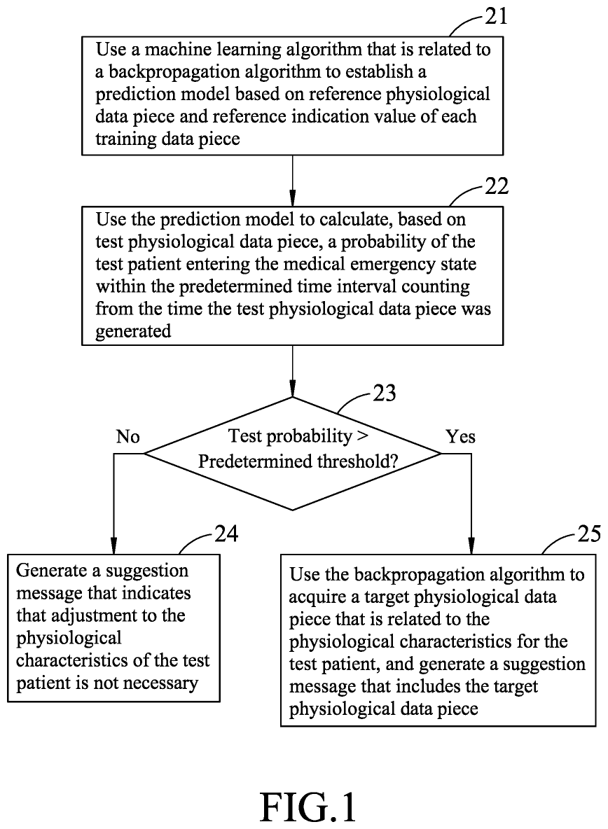

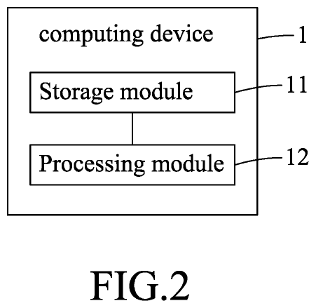

Referring to FIGS. 1 and 2, the first embodiment of a method of obtaining advice data of physiological characteristics for a test patient according to this disclosure is provided in order to lower the risk of the test patient entering a medical emergency state, or medical emergency state for short, and is implemented using a computing device 1 that includes a storage module 11, and a processing module 12 electrically connected to the storage module 11. In this embodiment, the computing device 1 may be realized as a desktop computer, a notebook computer, a cloud server, a supercomputer, or the like, and this disclosure is not limited in this respect. The storage module 11 may be, for example, a flash memory module, a hard disk drive, a solid-state drive, etc., and this disclosure is not limited in this respect. The processing module 12 may be, for example, a single-core processor, a multi-core processor, a dual-core mobile processor, a microprocessor, a microcontroller, a digital signal processor (DSP), a field-programmable gate array (FPGA), an application-specific integrated circuit (ASIC), a radio-frequency integrated circuit (RFIC), etc., but this disclosure is not limited in this respect.

The storage module 11 stores a plurality of training data pieces that are respectively related to and thus correspond to a plurality of reference patients, a test physiological data piece that is related to physiological characteristics of a test patient, and a set of predetermined rules that define reasonable physiological characteristics for human beings. Each of the training data pieces includes a reference physiological data piece that is related to physiological characteristics of the corresponding one of the reference patients and a reference indication value that indicates a truth of whether the corresponding one of the reference patients entered a medical emergency state within a predetermined time interval (e.g., thirty days) counting from the time the reference physiological data piece of the training data piece was generated. It is noted that the reference indication value may be provided from a database to serve as training material for machine learning. The physiological characteristics may include, but not limited to, age, sex, height, weight, body temperature, heart rate, diastolic blood pressure, systolic blood pressure, hemoglobin, white blood cell count, serum sodium, serum potassium, and so on. The medical emergency state may be predefined by medical professionals as desired, such as including any one of needing entering an intensive care unit, having the need to use specific medical equipment (e.g., extracorporeal membrane oxygenation (ECMO), dialysis machine, respirator, etc.), being in a life-threatening condition, and so on.

Referring to FIG. 1, the first embodiment includes steps 21-25.

In step 21, the processing module 12 uses a machine learning algorithm (e.g., a multilayer perceptron) that is related to a backpropagation algorithm to establish, based on the reference physiological data piece and the reference indication value of each of the training data pieces, a prediction model that uses a given physiological data piece related to physiological characteristics of a given patient to calculate a probability of the given patient entering the medical emergency state within the predetermined time interval counting from the time the given physiological data piece was generated.

In step 22, the processing module 12 makes the test physiological data piece serve as the given physiological data piece and uses the prediction model to calculate a test probability, which is an estimated probability of the test patient entering the medical emergency state within the predetermined time interval counting from the time the test physiological data piece was generated. As an example, assuming that the test physiological data piece indicates that the test patient is a male who is 50 years old, measures 170 cm in height and 130 kg in weight, and has a body temperature of 36.5° C., a heart rate of 100 bpm, a diastolic blood pressure of 120 mmHg, a systolic blood pressure of 150 mmHg, serum sodium of 150 mmol/L, and a hemoglobin of 17 g/dL, the processing module 12 may use the prediction model to calculate the probability (i.e., the test probability) of the test patient entering the medical emergency state within thirty days counting from the time these data were generated.

In step 23, the processing module 12 determines whether the test probability is greater than a predetermined threshold. The flow goes to step 24 when the determination is negative and goes to step 25 when otherwise. In this embodiment, the predetermined threshold may be, for example, 95%, but this disclosure is not limited in this respect.

In step 24, the processing module 12 generates a suggestion message that indicates that adjustment to the physiological characteristics of the test patient is not necessary. The suggestion message may indicate that the test patient is in a stable condition and has a lower probability of entering the medical emergency state because of variations in the physiological characteristics.

In step 25, the processing module 12 uses the backpropagation algorithm to acquire, based on a predetermined probability, the test physiological data piece and the prediction model, a target physiological data piece that is related to the physiological characteristics the test patient should achieve in order to lower the risk of entering the medical emergency state, and generates a suggestion message (the advice data) that includes the target physiological data piece. In this embodiment, the predetermined probability may be set to, for example, 0%, which represents that the test patient will not enter the medical emergency state within the predetermined time interval when his/her physiological characteristic is made to conform with the target physiological data piece. In this embodiment, the backpropagation algorithm may be represented by:

$$∇ x = \frac{\partial }{\partial x} H ( S , P ( x ) )$$

where ∇x represents a gradient to the target physiological data piece, H is a cross-entropy loss function, S represents the predetermined probability, P represents the prediction model, and X represents the test physiological data piece.

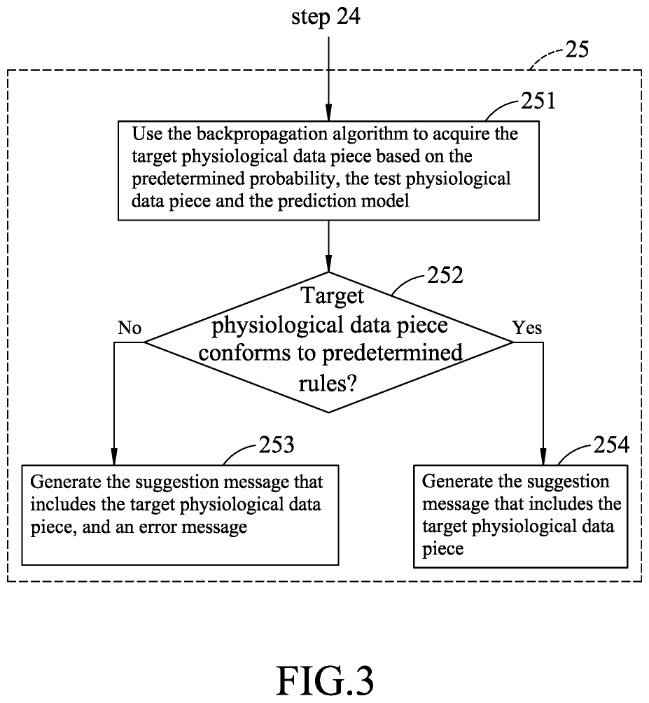

Referring to FIG. 3, step 25 includes sub-steps 251-254.

In sub-step 251, the processing module 12 uses the backpropagation algorithm to acquire the target physiological data piece based on the predetermined probability, the test physiological data piece, and the prediction model.

In sub-step 252, the processing module 12 determines, based on the test physiological data piece, whether the target physiological data piece conforms to the predetermined rules related to the physiological characteristics. The flow goes to sub-step 253 when determining that the target physiological data piece does not conform to any one of the predetermined rules and goes to sub-step 254 when otherwise. For example, the predetermined rules for the target physiological data piece may include, but not limited to, that target weight is not greater than 250 kg, that a target heart rate is not greater than 200 bpm, that a target diastolic blood pressure is not greater than 150 mmHg, that a target systolic blood pressure is not greater than 200 mmHg, that the target diastolic blood pressure is lower than the target systolic blood pressure, and that age, sex and a height included in the target physiological data piece are the same as those included in the test physiological data piece.

In sub-step 253, the processing module generates the suggestion message that includes the target physiological data piece and an error message indicating that the target physiological data piece does not conform to the set of predetermined rules so that a doctor/physician can evaluate the probability of the test patient entering the medical emergency state based on the suggestion message and determine a treatment/intervention for the test patient accordingly.

In sub-step 254, the processing module 12 generates the suggestion message that includes the target physiological data piece so that a doctor/physician can determine appropriate treatment/intervention for the test patient to improve the physiological characteristics of the test patient to meet the advice given in the target physiological data piece, thereby lowering the risk of the test patient entering the medical emergency state.

A second embodiment of the method of obtaining advice data of physiological characteristics for a test patient according to this disclosure is also implemented by the computing device 1, and differs from the first embodiment in that: (1) each of the training data pieces includes a reference symptom data piece that is unstructured data related to a symptom of a reference disease from which the corresponding one of the reference patients suffered and which results in physiological characteristics that are represented by the reference physiological data piece of the training data piece; (2) the storage module 11 further stores a test symptom data piece that is unstructured data related to a symptom of a test disease from which the test patient suffers and which results in physiological characteristics that are represented by the test physiological data piece; and (3) the storage module stores a first pre-processing model that is configured to convert unstructured text-related information into structured text-related information (e.g., bert-base-multilingual-cased, where “bert” stands for bidirectional encoder representations from transformers). For each of the training data pieces, the reference symptom data piece includes a reference chief complaint data piece that is text information summarizing a physiological condition of the corresponding one of the reference patients in relation to the reference disease, as detailed by the corresponding one of the reference patients (e.g., the reference patient describing how he/she felt when having the reference disease), and at least one reference illness data piece that is text information recording at least one previous illness experience (i.e., at least one illness experience prior to having the reference disease) of the corresponding one of the reference patients. In case the reference symptom data piece includes multiple reference illness data pieces, each of the reference illness data pieces corresponds to a respective one of the corresponding reference patient's previous illness experiences. The test symptom data piece includes a test chief complaint data piece that is text information summarizing a physiological condition of the test patient in relation to the test disease, as detailed by the test patient (e.g., the test patient describing how he/she felt when having the test disease), and at least one test illness data piece that is text information recording at least one previous illness experience (i.e., at least one illness experience prior to having the test disease) of the test patient. In case that the test symptom data piece includes multiple test illness data pieces, each of the test illness data pieces corresponds to a respective one of the test patient's previous illness experiences. Unstructured data/information means, for example, that the chief complaint data pieces that correspond to different patients (including the reference patients and the test patient) may include different content (e.g., reciting a headache for one patient, and reciting chest tightness for another patient), and/or that, in the illness data pieces that correspond to different patients, the ways of describing the previous illness experiences may be different. Structured data/information means that the data/information has a fixed structure; for example, each of the physiological data pieces is structured data since any physiological data piece would include data of age, sex, height, weight, body temperature, heart rate, diastolic blood pressure, systolic blood pressure, etc., of a corresponding patient.

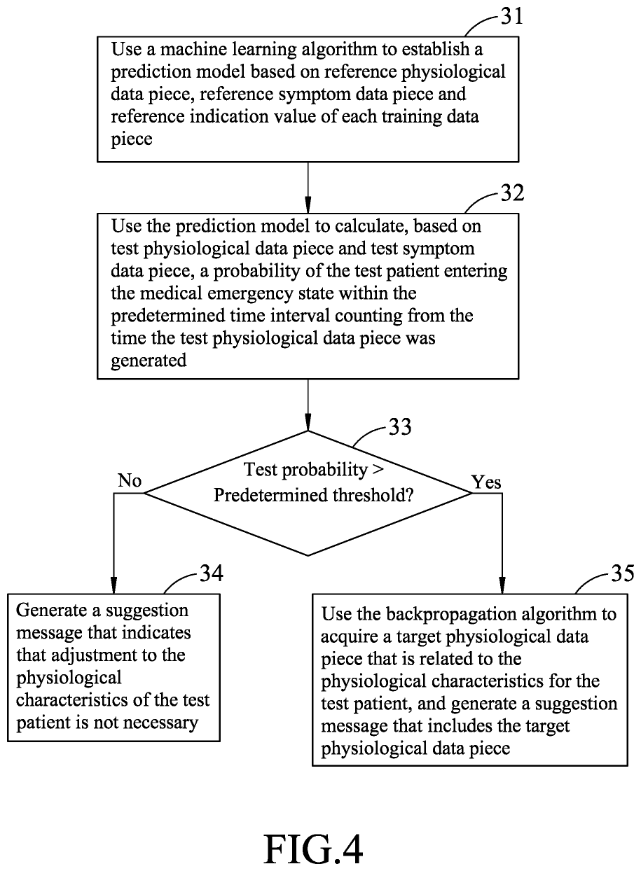

FIG. 4 is a flowchart that illustrates steps of the second embodiment, where steps 33-35 are similar to steps 23-25 of the first embodiment as shown in FIG. 1. The second embodiment differs from the first embodiment in steps 31 and 32.

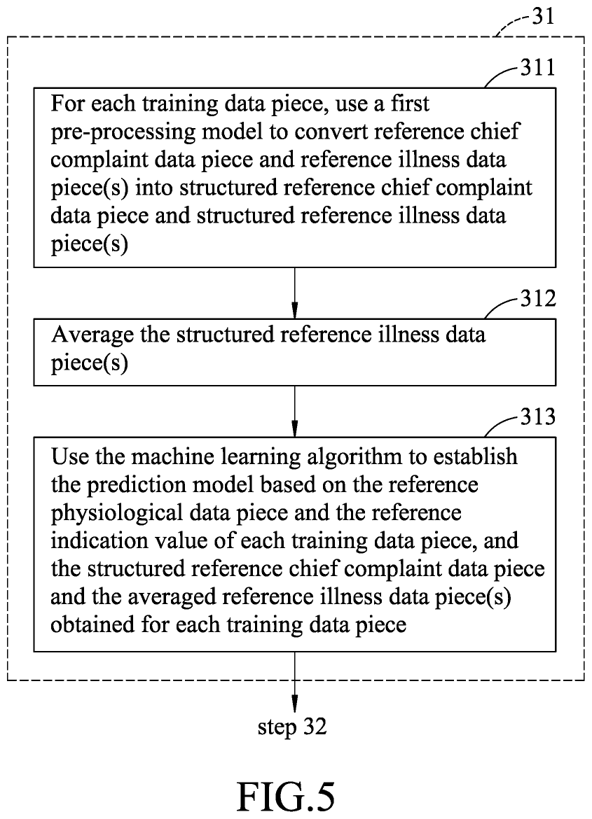

In step 31, the processing module 12 uses the machine learning algorithm to establish the prediction model based on the reference physiological data piece, the reference symptom data piece and the reference indication value of each of the training data pieces. Further referring to FIG. 5, step 31 includes sub-steps 311 to 313.

In sub-step 311, for each of the training data pieces, the processing module 12 uses the first pre-processing model to convert the (unstructured) reference chief complaint data piece and the at least one (unstructured) reference illness data piece of the reference symptom data piece of the training data piece into a structured reference chief complaint data piece and at least one structured reference illness data piece.

In sub-step 312, for each of the training data pieces, the processing module 12 averages at least one structured reference illness data piece obtained for the training data piece to obtain an averaged reference illness data piece. In case that the reference symptom data piece includes only one reference illness data piece, sub-step 312 may be omitted, and the reference illness data piece directly serves as the averaged reference illness data piece.

In sub-step 313, the processing module 12 uses the machine learning algorithm to establish the prediction model based on the reference physiological data piece and the reference indication value of each of the training data pieces, and the structured reference chief complaint data piece and the averaged reference illness data piece obtained for each of the training data pieces. In some embodiments, sub-step 312 may be omitted, and the processing module 12 uses the machine learning algorithm to establish the prediction model based on the reference physiological data piece and the reference indication value of each of the training data pieces, and the structured reference chief complaint data piece and all the structured reference illness data pieces obtained for each of the training data pieces.

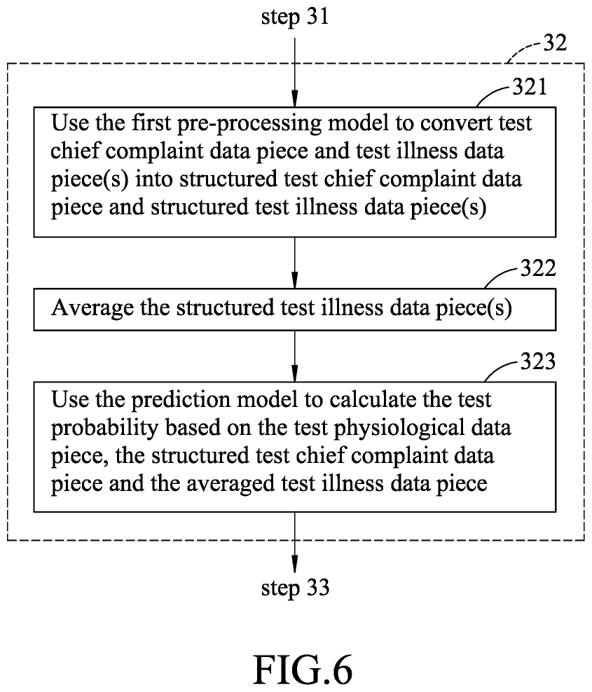

In step 32, the processing module 12 uses the prediction model to calculate the test probability based on the test physiological data piece and the test symptom data piece. Further referring to FIG. 6, step 32 includes sub-steps 321 to 323.

In sub-step 321, the processing module 12 uses the first pre-processing model to convert the (unstructured) test chief complaint data piece and the at least one (unstructured) test illness data piece into a structured test chief complaint data piece and at least one structured test illness data piece.

In sub-step 322, the processing module 12 averages the at least one structured test illness data piece to obtain an averaged test illness data piece. In case that the test symptom data piece includes only one test illness data piece, sub-step 322 may be omitted, and the test illness data piece directly serves as the averaged test illness data piece.

In sub-step 323, the processing module 12 uses the prediction model to calculate the test probability based on the test physiological data piece, the structured test chief complaint data piece and the averaged test illness data piece. In some embodiments, sub-step 322 may be omitted, and the processing module 12 uses the prediction model to calculate the test probability based on the test physiological data piece, the structured test chief complaint data piece and all the structured test illness data piece(s).

A third embodiment of the method of obtaining advice data of physiological characteristics for a test patient according to this disclosure is also implemented by the computing device 1, and differs from the second embodiment in that: (1) for each of the training pieces, the reference symptom data piece includes a reference image data piece that is graphical information related to the symptom of the reference disease, such as X-ray images, computed tomography (CT) images, etc.; (2) the test symptom data piece includes a test image data piece that is graphical information related to the symptom of the test disease; and (3) the storage module 11 stores a second pre-processing model that is configured to convert unstructured image-related information into structured image-related information (e.g., residual network (ResNet), which is an image-based feature extractor).

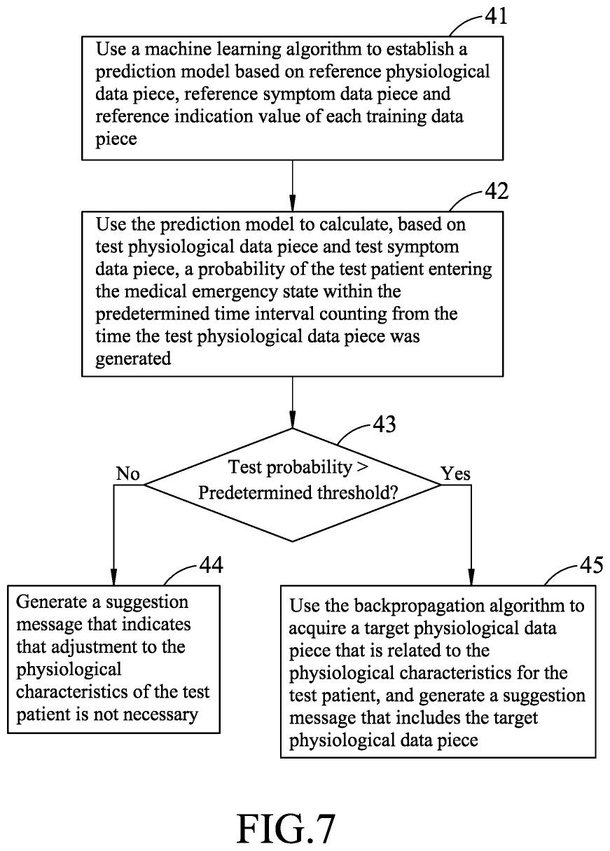

FIG. 7 is a flowchart that illustrates steps of the third embodiment, where steps 43-45 are similar to steps 33-35 of the second embodiment as shown in FIG. 4. The third embodiment differs from the second embodiment in steps 41 and 42.

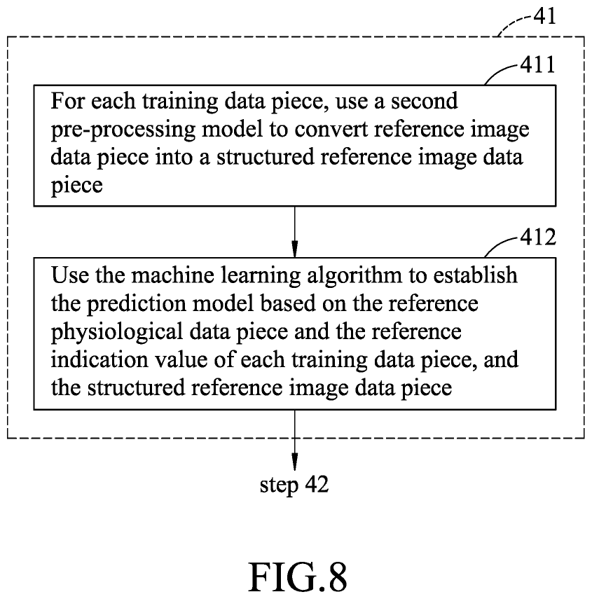

In step 41, the processing module 12 uses the machine learning algorithm to establish the prediction model based on the reference physiological data piece, the reference symptom data piece and the reference indication value of each of the training data pieces. Further referring to FIG. 8, step 41 includes sub-steps 411 and 412.

In sub-step 411, for each of the training data pieces, the processing module 12 uses the second pre-processing model to convert the (unstructured) reference image data piece of the training data piece into a structured reference image data piece.

In sub-step 412, the processing module 12 uses the machine learning algorithm to establish the prediction model based on the reference physiological data piece and the reference indication value of each of the training data pieces, and the structured reference image data piece obtained for each of the training data pieces.

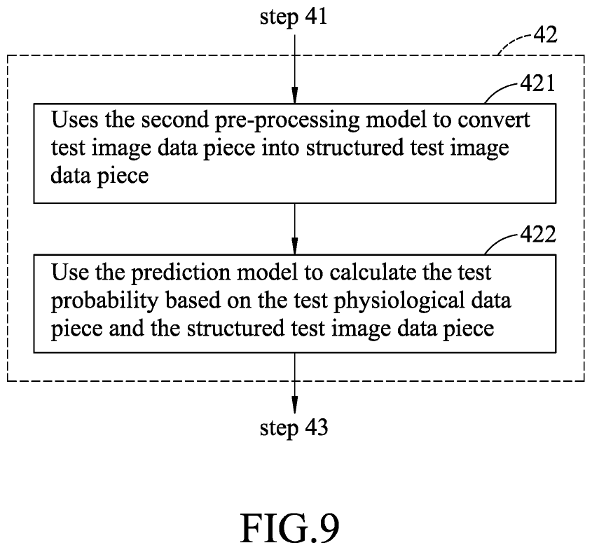

In step 42, the processing module 12 uses the prediction model to calculate the test probability based on the test physiological data piece and the test symptom data piece. Further referring to FIG. 9, step 42 includes sub-steps 421 and 422.

In sub-step 421, the processing module 12 uses the second pre-processing model to convert the (unstructured) test image data piece into a structured test image data piece.

In sub-step 422, the processing module 12 uses the prediction model to calculate the test probability based on the test physiological data piece and the structured test image data piece.

It is noted that, in the third embodiment, the reference symptom data piece of each of the training data pieces includes only the reference image data piece, and the test symptom data piece includes only the test image data pieces. In some embodiments, the reference symptom data piece of each of the training data pieces may include not only the reference image data piece as introduced in the third embodiment, but also the reference chief complaint data piece and the reference illness data piece(s) as introduced in the second embodiment, and the test symptom data piece may include not only the test image data piece as introduced in the third embodiment, but also the test chief complaint data piece and the test illness data piece(s) as introduced in the second embodiment. In such a scenario, the storage module 11 stores both the first pre-processing model and the second pre-processing model, so the processing module 12 can convert the unstructured reference image data piece, reference chief complaint data piece, reference illness data piece(s), test image data piece, test chief complaint data piece and test illness data piece(s) into their structured counterparts, namely, the structured reference image data piece, the structured reference chief complaint data piece, the structured reference illness data piece(s), the structured test image data piece, the structured test chief complaint data piece and the structured test illness data piece(s). The processing module 12 uses the machine learning algorithm to establish the prediction model based on the reference physiological data piece and the reference indication value of each of the training data pieces, and the structured reference chief complaint data piece, the averaged reference illness data piece and the structured reference image data piece obtained for each of the training data pieces. Then, the processing module 12 uses the prediction model to calculate the test probability based on the test physiological data piece, the structured test chief complaint data piece, the averaged test illness data piece and the structured test image data piece.

In summary, the embodiments of the method of obtaining advice data of physiological characteristics for a test patient according to this disclosure use the training data pieces to build the prediction model, and use the prediction model to calculate the probability of the test patient entering the medical emergency state based on the test physiological data piece (and the test symptom data piece in some embodiments). Upon determining that the probability is greater than the predetermined threshold, the backpropagation algorithm is used to acquire the target physiological data piece for the test patient based on the predetermined probability and the prediction model, and the suggestion message that includes the target physiological data piece. In other words, the embodiments use big data relating to previous medical experiences to generate the suggestion message rapidly, so as to assist a doctor/physician to efficiently make appropriate decisions on subsequent treatments/interventions within the golden period, thereby lowering the likelihood for the test patient to enter the medical emergency state.

In the description above, for explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of obtaining advice data of physiological characteristics for a test patient in order to lower risk of the test patient entering a medical emergency state, said method comprising steps of:
A) providing a plurality of training data pieces to a computing device, wherein the training data pieces are respectively related to a plurality of reference patients, and each of the training data pieces includes:
a reference physiological data piece that is related to physiological characteristics of the corresponding one of the reference patients, and
a reference indication value that indicates a truth of whether the corresponding one of the reference patients entered the medical emergency state within a predetermined time interval counting from the time the reference physiological data piece of the training data piece was generated;
B) by the computing device, using a machine learning algorithm that is related to a backpropagation algorithm to establish, based on the reference physiological data piece and the reference indication value of each of the training data pieces, a prediction model that uses a given physiological data piece that is related to physiological characteristics of a given patient to calculate a probability of the given patient entering the medical emergency state within the predetermined time interval counting from the time the given physiological data piece was generated;
C) providing a test physiological data piece to the computing device, wherein the test physiological data piece is related to the physiological characteristics of the test patient;
D) by the computing device, making the test physiological data piece serve as the given physiological data piece, and using the prediction model to calculate a test probability, which is an estimated probability of the test patient entering the medical emergency state within the predetermined time interval counting from the time the test physiological data piece was generated;
E) by the computing device, determining whether the test probability is greater than a predetermined threshold; and
F) by the computing device, upon determining that the test probability is greater than the predetermined threshold, using the backpropagation algorithm to acquire, based on a predetermined probability, the test physiological data piece and the prediction model, a target physiological data piece that is related to the physiological characteristics the test patient should achieve in order to lower risk of entering the medical emergency state, and generating a first suggestion message that includes the target physiological data piece and that serves as the advice data.

2. The method of claim 1, further comprising a step of:
G) by the computing device, upon determining that the test probability is not greater than the predetermined threshold, generating a second suggestion message that indicates that adjustment to the physiological characteristics of the test patient is not necessary.

3. The method of claim 1, wherein step F) includes sub-steps of:
F-1) determining, based on the test physiological data piece, whether the target physiological data piece conforms to a set of predetermined rules that are related to the physiological characteristics;
F-2) upon determining that the target physiological data piece does not conform to the set of predetermined rules, generating the first suggestion message that includes the target physiological data piece, and an error message indicating that the target physiological data piece does not conform to the set of predetermined rules; and
F-3) upon determining that the target physiological data piece conforms to the set of predetermined rules, generating the first suggestion message that includes the target physiological data piece.

4. The method of claim 1, wherein each of the training data pieces further includes a reference symptom data piece that is unstructured data related to a symptom of a reference disease from which the corresponding one of the reference patients suffered and which results in physiological characteristics that are represented by the reference physiological data piece of the training data piece, and the prediction model is established further based on the reference symptom data piece of each of the training data pieces in step B); and
wherein the test probability is calculated further based on a test symptom data piece that is unstructured data related to a symptom of a test disease from which the test patient suffers and which results in physiological characteristics that are represented by the test physiological data piece.

5. The method of claim 4, wherein, for each of the training data pieces, the reference symptom data piece includes a reference chief complaint data piece that is text information summarizing a physiological condition of the corresponding one of the reference patients in relation to the reference disease, and a reference illness data piece that is text information recording a previous illness experience of the corresponding one of the reference patients; and
wherein step B) includes sub-steps of:
B-1) for each of the training data pieces, using a pre-processing model that is configured to convert unstructured text-related information into structured text-related information to convert the reference chief complaint data piece and the reference illness data piece of the training data piece into a structured reference chief complaint data piece and a structured reference illness data piece; and
B-2) using the machine learning algorithm to establish the prediction model based on the reference physiological data piece and the reference indication value of each of the training data pieces, and the structured reference chief complaint data piece and the structured reference illness data piece obtained for each of the training data pieces.

6. The method of claim 4, wherein, for each of the training data pieces, the reference symptom data piece includes a reference chief complaint data piece that is text information summarizing a physiological condition of the corresponding one of the reference patients in relation to the reference disease, and a plurality of reference illness data pieces that are text information recording multiple previous illness experiences of the corresponding one of the reference patients; and
wherein step B) includes sub-steps of:
B-1) for each of the training data pieces, using a pre-processing model that is configured to convert unstructured text-related information into structured text-related information to convert the reference chief complaint data piece and the reference illness data pieces of the training data piece into a structured reference chief complaint data piece and a plurality of structured reference illness data pieces;
B-2) for each of the training data pieces, averaging the structured reference illness data pieces obtained for the training data piece to obtain an averaged reference illness data piece; and
B-2) using the machine learning algorithm to establish the prediction model based on the reference physiological data piece and the reference indication value of each of the training data pieces, and the structured reference chief complaint data piece and the averaged reference illness data piece obtained for each of the training data pieces.

7. The method of claim 4, wherein the test symptom data piece includes a test chief complaint data piece that is text information summarizing a physiological condition of the test patient in relation to the test disease, and a test illness data piece that is text information recording a previous illness experience of the test patient; and
wherein step D) includes sub-steps of:
D-1) using a pre-processing model that is configured to convert unstructured text-related information into structured text-related information to convert the test chief complaint data piece and the test illness data piece into a structured test chief complaint data piece and a structured test illness data piece; and
D-2) using the prediction model to calculate the test probability based on the test physiological data piece, the structured test chief complaint data piece and the structured test illness data piece.

8. The method of claim 4, wherein the test symptom data piece includes a test chief complaint data piece that is text information summarizing a physiological condition of the test patient in relation to the test disease, and a plurality of test illness data pieces that are text information recording multiple previous illness experiences of the test patient; and
wherein step D) includes sub-steps of:
D-1) using a pre-processing model that is configured to convert unstructured text-related information into structured text-related information to convert the test chief complaint data piece and the test illness data pieces into a structured test chief complaint data piece and a plurality of structured test illness data pieces;
D-2) averaging the structured test illness data pieces to obtain an averaged test illness data piece; and
D-3) using the prediction model to calculate the test probability based on the test physiological data piece, the structured test chief complaint data piece and the averaged test illness data piece.

9. The method of claim 4, wherein, for each of the training data pieces, the reference symptom data piece includes a reference image data piece that is graphical information related to the symptom of the reference disease; and
wherein step B) includes sub-steps of:
B-1) for each of the training data pieces, using a pre-processing model that is configured to convert unstructured image-related information into structured image-related information to convert the reference image data piece of the training data piece into a structured reference image data piece; and
B-2) using the machine learning algorithm to establish the prediction model based on the reference physiological data piece and the reference indication value of each of the training data pieces, and the structured reference image data piece obtained for each of the training data pieces.

10. The method of claim 4, wherein the test symptom data piece includes a test image data piece that is graphical information related to the symptom of the test disease; and
wherein step D) includes sub-steps of:
B-1) using a pre-processing model that is configured to convert unstructured image-related information into structured image-related information to convert the test image data piece into a structured test image data piece; and
B-2) using the prediction model to calculate the test probability based on the test physiological data piece and the structured test image data piece.
